(19) United States
(12) Patent Application Publication
(10) Pub. No.: US 2022/0143126 A1
(43) Pub. Date: May 12, 2022

(54) SYNERGISTIC COMBINATION OF PHYTOACTIVES

(71) Applicant: Shankaranarayanan JEYAKODI, Telangana, Hyderabad (IN)

(72) Inventors: Shankaranarayanan JEYAKODI, Telangana Hyderabad (IN); Arunkanth KRISHNAKUMAR, Telangana Hyderabad (IN); Kalyan JANJANAM CHAKRAVARTHY, Telangana Hyderabad (IN)

(21) Appl. No.: 17/434,422

(22) PCT Filed: Mar. 28, 2020
(86) PCT No.: PCT/IN2020/050295

§ 371 Date: Aug. 27, 2021

(30) Foreign Application Priority Data
Mar. 31, 2019 (IN) 201941012952

(51) Int. Cl. A61K 36/9066; A61K 31/12; A61K 31/216; A61K 31/353

(52) U.S. Cl. CPC A61K 36/9066; A61K 31/353; A61K 31/216; A61K 31/12

(57) ABSTRACT

The present invention relates to a combination of phytoactives comprising plant extracts that deliver curcumin, chlorogenic acid and/or catechin. The invention also relates to a process for preparing such combination with enhanced therapeutic and pharmacological effect. The phytoactives combination is used for enhancing endurance and stamina, for significantly reducing cortisol levels thus reducing stress, for elevating hydrolysis of adenosine triphosphate (ATP), for improving blood flow, for quick recovery from sports injury, for better weight and fat loss compared to individual actives, for increasing and strengthening muscles and for increasing maximum oxygen uptake during incremental exercise.

### CROSS REFERENCE TO RELATED APPLICATION

This application claims priority to co-pending Indian Provisional Patent Application Serial Number 201941012952 filed on Mar. 31, 2019. This application is incorporated herein by reference, in its entirety.

### TECHNICAL FIELD OF THE INVENTION

The present invention relates to a combination of phytoactives comprising plant extracts that deliver curcumin, chlorogenic acid and/or catechin. The invention also relates to a process for preparing such combination with enhanced therapeutic and pharmacological effect.

### BACKGROUND OF THE INVENTION

Curcumin a phytopolyphenol pigment isolated from the plant Curcuma longa, commonly known as turmeric, has a variety of pharmacologic properties. Curcumin is obtained from Curcuma longa (Zingiberaceae), a perennial herb widely cultivated in tropical regions of Asia. Its rhizome is extensively used for imparting color and flavor to food. Current traditional Indian medicine claims the use of its powder, turmeric, against a wide variety of diseases. Extensive scientific research on curcumin has demonstrated a wide spectrum of therapeutic effects which range from anti-inflammatory, wound healing, antispasmodic, anticoagulant, antitumor activities, etc. Studies on curcumin relating to absorption, distribution, metabolism and excretion have revealed poor absorption and rapid metabolism of curcumin which severely curtails its bioavailability.

Green coffee beans are coffee seeds (beans) of Coffee fruits that have not yet been roasted. Green coffee beans have a higher level of chlorogenic acid compared to regular, roasted coffee beans because during the roasting process the amount of chlorogenic acid gets reduced in coffee beans. Chlorogenic acid in green coffee is known to have health benefits. Most of the prospective epidemiologic studies suggested that long-term consumption of coffee and decaffeinated coffee can reduce the risk of diabetes. One of the studies found that the intake of caffeinated coffee, decaffeinated coffee, and caffeine were independently associated with weight loss. It is proven that long-term coffee consumption has an ability to help people achieve weight loss, and this further contributes towards reducing the risk of diabetes (Greenberg, J. A., Boozer, C. N., and Geliebter, A. Coffee, diabetes, and weight control. Am. J. Clin. Nutr. 2006; 84(4):682-693). Further, it is known to a person skilled that the Green coffee bean extract improves obesity, decreases mRNA expression levels of adipogenesis and adipocyte, helps in non-alcoholic fatty liver disease, reduces blood pressure and due to its anti-oxidant property shows favorable response in patients with certain type of cancer and helps in preventing signs of skin aging.

Green tea, obtained from Camellia sinensis, is not fermented but produced by steaming fresh leaves at high temperature. During the process, levels of polyphenols are retained which is in turn responsible for the benefits of green tea. Green tea is consumed to improve—mental alertness and thinking, depression, non-alcoholic fatty liver disease (NAFLD), inflammatory bowel disease (ulcerative colitis or Crohn's disease), weight loss and also treat stomach disorders, vomiting, diarrhea, headache, and bone loss (osteoporosis).

It is well known that curcumin exhibits poor bioavailability. The major reasons attributed to the low bioavailability of curcumin are poor absorption, rapid metabolism, and rapid systemic elimination. As per Wahlstrom and Blennow report published in BCPT; Volume 43, Issue 2 after oral administration of 1 g/kg curcumin to rats, more than 75% of curcumin was excreted in faeces and negligible amount was detected in urine.

The study conducted by Shobha et al demonstrated that concomitant administration of curcumin with piperine produced 150% increase in bioavailability in rats and 2000% increase in man (Shoba et al., 1998). Other ways to improve the bioavailability of curcumin is by making curcumin nanoparticles (Bisht et al., 2007), liposomes (Li et al., 2005), micelles and phospholipid complexes (Maiti et al., 2007) (Marczylo et al., 2007). The possible advantages attributed to such formulations are (a) provide longer circulation; (b) increase the cellular permeability and (c) induce resistance to metabolic processes.

Apart from published literature there are some patent documents that describe the methods for enhancing bioavailability of the food extracts.

KR101886802B1 and KR20130036631A disclose catechin bioavailability enhancer compositions. As per the patent description, catechin may be included in the composition in the form of an extract of Camellia sinensis. Further the patent describes that when the enhancer includes an acid and a sugar alcohol, the bioavailability of the catechin is increased, thus resulting in better weight loss, good body fat reduction and anti-oxidation effect.

JP2007060905A, JP2007314440A and JP2007063137A all disclose curcumin composition as health food, for physical fitness enhancement and as anti-obesity drug. The applications disclose that the composition further comprises one or more of ouren, oubaku, maor, forest, forest, green tea, oolong tea, black tea, coffee, quercusiae, teensa, agar, capsicum, ginger and pepper.

US20170157194 discloses a plant extract composition for reducing body fat and body weight which comprises green tea extract and a turmeric extract.

The reasons for reduced bioavailability of any agent within the body may be attributed to low intrinsic activity, poor absorption, higher rate of metabolism, inactivity of metabolic products and/or rapid elimination from the body. Although prior art applications disclose compositions comprising curcumin or combination with green tea or coffee, none of these applications disclose synergism between the components which is achieved by the present formulation.

The main reason for poor absorption of curcumin is the poor solubility in water. Thus, there is a need to develop alternate formulation approaches for lipophilic nutrients so as to design dosage forms with enhanced stability and solubility thereby providing enhanced bioavailability.

### OBJECT OF THE INVENTION

An object of the present invention is to provide a combination of phytoactives.

Another object of the present invention is to provide a combination comprising plant extracts that deliver curcumin, chlorogenic acid and/or catechin.

Yet another object of the present invention is to provide a combination of phytoactives that increases the bioavailability of curcumin.

Another object of the present invention is to provide a combination that is prepared by encapsulating curcumin, chlorogenic acid and/or catechin.

Yet another object of the present invention is to provide a composition comprising active ingredient in the range from 1%-95% w/w preferably 20%-50% w/w and encapsulating agent in the range from 5%-90% w/w preferably 30%-70% w/w.

Another object of the present invention is to provide a process for preparing the combination which comprises encapsulating curcumin, chlorogenic acid and/or catechin to obtain powder or granules.

Yet another object of the present invention is to provide a composition of phytoactives that can be delivered by oral route.

Another object of the present invention is to provide a combination that delivers the active ingredients in the range of—Curcumin: 10 mg to 500 mg/day; Chlorogenic acid: 10 mg to 800 mg/day and Catechin: 5 mg to 1000 mg/day.

Still another object of the present invention is to provide a combination of phytoactives for enhancing endurance and stamina, for significantly reducing cortisol levels thus reducing stress, for elevating hydrolysis of adenosine triphosphate (ATP), for improving blood flow, for quick recovery from sports injury, for better weight and fat loss compared to individual actives, for increasing and strengthening muscles and for increasing maximum oxygen uptake during incremental exercise.

### SUMMARY OF THE INVENTION

An embodiment of the present invention is to provide a combination of phytoactives.

Another embodiment of the present invention is to provide a combination comprising plant extracts that deliver curcumin, chlorogenic acid and/or catechin.

Yet another embodiment of the present invention is to provide a combination of phytoactives that increases the bioavailability of curcumin.

Another embodiment of the present invention is to provide a combination that is prepared by encapsulating curcumin, chlorogenic acid and/or catechin.

Yet another embodiment of the present invention is to provide a composition comprising active ingredient in the range from 1%-95% w/w preferably 20%-50% w/w and encapsulating agent in the range from 5%-90% w/w preferably 30%-70% w/w.

Another embodiment of the present invention is to provide a process for preparing the combination which comprises encapsulating curcumin, chlorogenic acid and/or catechin to obtain powder or granules.

Yet another embodiment of the present invention is to provide a combination that delivers the active ingredients in the range of—Curcumin: 10 mg to 500 mg/day; Chlorogenic acid: 10 mg to 800 mg/day and Catechin: 5 mg to 1000 mg/day.

Still another embodiment of the present invention is to provide a combination of phytoactives for enhancing endurance and stamina, for significantly reducing cortisol levels thus reducing stress, for elevating hydrolysis of adenosine triphosphate (ATP), for improving blood flow, for quick recovery from sports injury, for better weight and fat loss compared to individual actives, for increasing and strengthening muscles and for increasing maximum oxygen uptake during incremental exercise.

### DETAILED DESCRIPTION OF THE INVENTION

Although prior art applications disclose compositions comprising curcumin or combination with green tea or coffee, none of these applications disclose synergism between the components which is achieved by the present formulation.

It should be appreciated that the compositions containing one or more items selected from the group consisting of chlorogenic acid and/or catechin combined with curcumin worked synergistically by improving the therapeutic and pharmacological effect of the active ingredients.

The term “synergy” or “synergistic” as used herein, unless otherwise specified, refers to the interaction of two or more compounds so that their combined effect is greater than the additive sum of their individual effects.

Synergistic effect of the composition is shown by enhancement in solubility and stability of the active ingredients. Through the present composition the stability and water solubility of curcumin is increased, antioxidant potential of catechin is enhanced and metabolism of chlorogenic acid is reduced.

Lipophilic nutrients like curcumin has reduced bioavailability within the body due to low intrinsic activity, poor absorption, higher rate of metabolism, inactivity of metabolic products and/or rapid elimination from the body. Thus, there is a need to develop alternate formulation approaches for lipophilic nutrients so as to design dosage forms with enhanced stability and solubility thereby providing enhanced bioavailability.

In the food-processing field, encapsulation techniques have been widely used to protect food extracts against deterioration or volatile losses. The protective mechanism is to form a membrane, the wall system, around droplets or particles of the encapsulated material, the core. Encapsulation not only protects against losses and chemical changes, but also enables production in the form of powdered products with new properties.

In an embodiment, the combination of the present invention comprises encapsulating the plant extracts that deliver curcumin, chlorogenic acid and/or catechin.

In yet another embodiment, the encapsulation process of the present invention enhances the stability and solubility of curcumin which further leads to enhanced bioavailability.

In the present invention, Curcuma longa (commonly known as turmeric) is used as the source for curcumin; Green coffee bean i.e. coffee seeds (beans) of Coffee fruits that have not yet been roasted is used as the source of chlorogenic acid and Green tea (obtained from Camellia sinensis that is not fermented but produced by steaming fresh leaves at high temperature) is used as source for catechin.

The curcumin, chlorogenic acid and/ or catechin is extracted from the sources mentioned above by using conventional or non-conventional extraction methods that are known to a person skilled in art.

According to one embodiment, the combination of the present invention is prepared by a process comprising plant extracts that deliver curcumin, chlorogenic acid and/or catechin; encapsulating agent(s) and solvent(s).

The ratio of components used in the composition of the present invention are as follows: active ingredient ranges from 1%-95% w/w preferably 20%-50% w/w and encapsulating agent ranges from 5%-90% w/w preferably 30%-70% w/w.

The encapsulating agent(s) used in the composition of the present invention is selected from modified starch, gum acacia, dextrin, dried glucose syrup, starch, maltodextrin or combinations thereof.

Preferably the encapsulating agent used in the present invention is modified starch and/or maltodextrin.

The solvent(s) used in the present invention are selected from the group such as, but not limited to, acetone, hexane, ethyl acetate, isopropyl alcohol, ethanol, dichloromethane, methanol, and a mixture thereof.

Preferably the solvent(s) used in the present invention are acetone, ethanol, dichloromethane, isopropyl alcohol. More preferably dichloromethane, ethanol and isopropyl alcohol.

In yet another embodiment, the combination of the present invention delivers the active ingredients in the range of—Curcumin: 10 mg to 500 mg/day; Chlorogenic acid: 10 mg to 800 mg/day and Catechin: 5 mg to 1000 mg/day.

The combination of phytoactives of the present invention is prepared by a process comprising:
(i) dissolving the active ingredients in a solvent or mixture of solvents to form a clear solution;
(ii) dissolving the encapsulating agent in water
(iii) adding step (i) solution in step (ii) under stirring;
(iii) removing the solvent by evaporation; and
(iv) drying the residue to obtain powder or granules.

In an embodiment the process of the present invention further comprises of suitable pharmaceutical and/or nutraceutical excipient, but not limited to co-solvent, glidant, binder, thickener, surfactant or combination thereof.

In a preferred embodiment, the drying of residue in the process of present invention is carried out by using bottom spray, top spray fluid bed processor or by tangential spray, top spray Flex Stream process.

In an embodiment, the combination of the present invention is formulated for delivery by the oral route.

The combination of the present invention is dispensed in dosage forms selected from powder, pellets, beadlets, granules, tablet, capsules, buccal films and the like.

In a further embodiment, the combination of phytoactives of the present invention enhances endurance and stamina, significantly reduces cortisol levels thus stress, elevates hydrolysis of adenosine triphosphate (ATP), improves blood flow, helps in quick recovery from sports injury, promotes better weight and fat loss compared to individual actives, increases muscle strength and also increases maximum oxygen uptake during incremental exercise.

The composition of the present invention which is a combination of curcumin, chlorogenic acid and/or catechin is planned to be tested for efficacy in rats. The details of the study are captured below:

| | |
|---|---|
| Object of the study | To evaluate the efficacy in Muscle injury model |
| Type of Study | Efficacy |
| Animal Strain | Male Sprague-Dawley Rats |
| Body weight of animals | 200-250 g |
| Study Duration | 15 days |
| Acclimatization period | 5 days |
| Total duration of the study | 22 days |
| Number of animals/Time Point | 6 |
| Groups | 4 Groups |
| | Group 1: Normal Control |
| | Group 2: Muscle Injury Control |
| | Group 3: Test Item Dose 1 |
| | Group 4: Reference Control |
| Total number of animals | 24 |
| Route of administration | Oral |
| Frequency of Test product | Once daily |
| administration | |

All the rats will receive their respective treatments from day 1 of the study till 14 days. Control group animals will receive saline. After 7 day of the treatment, Barium Chloride will be injected intramuscularly in the quadriceps of the left extremity in the rats to induce muscle injury, causing total degeneration of the muscle. In the rats of control group (Group 1), 200 μl of saline solution will be injected. At seven days after Barium Chloride injection, rats will be sacrificed and samples will be obtained to determine the effects of Barium Chloride-induced muscle injury.

Before sacrificing the animals, blood will be collected to estimate TNF-α and IL-1β as an indicator of muscle injury. The muscled from the site of injection will be collected and divided into two portions. One portion will be fixed in 10% neutral buffered formalin for further histopathological evaluation while second portion of muscle will be homogenized in suitable buffer to estimate the oxidative stress.

Further, a prospective, double blind, reference controlled randomised interventional study to evaluate the efficacy and safety of the phytoactive combination of the present invention as a supplement in accelerating healing from sports injury is to be soon initiated.

The details of the Interventional study are as follows:

| Study Title | Prospective, double blind, reference controlled randomised | |
|---|---|---|
| | interventional study to evaluate the efficacy and safety of phytoactives | |
| | combination of the present invention as a supplement in accelerating | |
| | healing from sports injury. | |
| Investigational Product | Phytoactives combination of the present invention | |
| Comparator | Physical Mixture of Curcumin Extract and Green Coffee Bean Extract | |
| Phase | Interventional Study | |
| Indication | Muscular injury caused due to high endurance sports. | |
| Study Design | Prospective, double blind, randomised interventional study | |
| Study Duration | 3 months | |
| Treatment Duration | 1 week | |
| Sample Size | Minimum 12(1:1) | |
| Dose/Dosage regimen | Phytoactives combination of the present | |
| | invention - 500 mg BID | |
| | Physical Mixture of Curcumin Extract and Green | |
| | Coffee Bean Extract- 500 mg BID | |
| Objective | Primary: | |
| | To evaluate the efficacy of Investigational Product as a supplement in | |
| | accelerating healing from injury caused due to high endurance sports. | |
| | This will be assessed through the following 3 parameters: | |
| | 1. | Recovery from muscle damage due to sports injury |
| | 2. | Increased Endurance |
| | 3. | Reduce Inflammation |
| | 4. | |
| | Secondary: | |
| | To evaluate the safety of Investigational Product during the entire | |
| | study duration | |
| Endpoints | Primary (Efficacy): | |
| | Change in following biomarker test from baseline to end of treatment: | |
| | 1. | Recovery from muscle damage due to sports injury |
| | | Creatine Kinase |
| | | Myoglobin |
| | | Blood Urea Nitrogen |
| | | Lactate Dehydrogenase |
| | 2. | Increased Endurance |
| | | Serum ferratin |
| | | Total iron concentration |
| | | Transferrin |
| | | Sodium |
| | | Physical tests: squats, pushups, core |
| | 3. | Reduce Inflammation |
| | | CBC |
| | Secondary (Safety): | |
| | 1. | Occurrence of any AE or SAE during the study. |
| | 2. | Occurrence of any clinically significant abnormality as per the |
| | | Principal Investigator in the following safety marker test: |
| | | Urine-routine |
| | | Random Blood Sugar |
| | | Heart Rate |
| | | POMS questionnaire |
| | | Respiratory Rate |
| Inclusion Criteria | 1. | Male or Female aged 18-50 years |
| | 2. | Subjects willing to comply to study procedure and sign written informed consent |
| | 3. | Subjects with acute muscular injury like muscle strain (grade 1-3), |
| | | Cramp, acute compartment syndrome. |
| Exclusion Criteria | 1. | Subjects requiring surgery for the injury |
| | 2. | Subjects with open wounds |
| | 3. | Subjects with injury involving skeletal system or deep soft tissue injury |
| | 4. | Subjects already on medication for any sports injury |
| | 5. | Subjects on steroids or ergogenic users |
| | 6. | Subjects with uncontrolled systemic disease since past 3 months. |
| | 7. | Subject with neurological disorders |
| | 8. | Subjects on drug abuse, narcotic abuse, alcoholism or smoking |
| | 9. | Breast feeding women |
| | 10. | Pregnant women |
| | 11. | Subject planning to conceive |
| | 12. | Subjects participating in another clinical trial or subjects |
| | | participated in any clinical trial within last 6 months. |
| | 13. | Any condition that in the opinion of the investigator does not justify the subject's inclusion for the study. |

| | |
|---|---|
| Statistical Methodology | ANOVA and suitable statistical method will be followed. |

From the details given above it can be observed that the composition of the present invention is not a mere admixture resulting in a composition which having the aggregation of the properties of the components used but a composition formed by the synergistic activities of the components used.

The nature of the invention, its objects and advantages are explained hereunder in greater detail in relation to non-limiting exemplary embodiments.

The following examples are for the purpose of illustration of the invention only and are not intended in any way to limit the scope of the present invention.

### EXAMPLE 1

| Sr. No | Name of Ingredient | Qty for 100 g |
|---|---|---|
| 1 | Curcumin Extract | 12.63 |
| 2 | Green coffee bean extract | 53.33 |
| 3 | Polyethylene glycol 6000 | 24.00 |
| 4 | Maltodextrin | 10.04 |
| 5 | Isopropyl alcohol | 25.00 |

24.00 g of Polyethylene glycol 6000 was melted at temperature of 75° C. and 12.63 g of curcumin extract was added to it and dissolved. The mixture was cooled upto 35° C. and 25.00 g isopropyl alcohol was added. To this mixture 53.33 g of Green coffee bean extract and 10.04 g of maltodextrin were added. The temperature was gradually raised and mixing was continued till all solvent had evaporated. After evaporation the solid mass obtained was cooled and milled to obtain uniform granules.

### EXAMPLE 2

| Sr. No | Name of Ingredient | Qty for 100 g |
|---|---|---|
| 1 | Curcumin Extract | 2.10 |
| 2 | Green Coffee Bean Extract | 17.77 |
| 3 | Ethanol | 50.0 |
| 4 | Methylene Dichloride | 70.0 |
| 5 | Modified Starch | 77.13 |
| 6 | Tween 80 | 3.00 |
| 7 | Purified Water | 180.0 |

2.10 g of curcumin extract was dissolved in mixture of 50 g ethanol and 70 g of methylene dichloride. 77.13 g modified starch and 3.00 g of Tween 80 was dissolved in 180 g of hot purified water at 50° C. The solvent phase was added slowly to the aqueous phase using both a high rate of mixing and a high shear force mixer. After the addition, the emulsion temperature was maintained at 50° C. and 17.77 g of green coffee bean extract was added and high speed shear mixing was continued for 15 min. The temperature was gradually raised and mixing was continued until all solvent was evaporated. During the evaporation procedure, distilled water was added to the emulsion to maintain a suitable viscosity. After all the ethanol and methylene dichloride was removed, the distilled water was added and thoroughly admixed with the emulsion to achieve an emulsion. The emulsion was then sprayed using spray dryer to obtain a powder.

### EXAMPLE 3

| Sr. No | Name of Ingredient | Qty for 100 g |
|---|---|---|
| 1 | Curcumin Extract | 11.52 |
| 2 | Green Coffee Bean Extract | 53.32 |
| 3 | Ethanol | 129.0 |
| 4 | Methylene Dichloride | 300.0 |
| 5 | Modified Starch | 31.16 |
| 6 | Tween 80 | 4.00 |
| 7 | Purified Water | 100.0 |

11.52 g of curcumin extract was dissolved in a mixture of 129.0 g ethanol and 300.0 g of methylene dichloride. 31.16 g modified starch and 4.00 g Tween 80 was dissolved in 100 g of hot purified water at 50° C. The solvent phase was added slowly to the aqueous phase using both a high rate of mixing and a high shear force mixer. After the addition was completed, the emulsion temperature was maintained at 50° C. and 53.32 g of green coffee bean extract was added. High speed shear mixing was continued for 15 min, temperature was gradually raised and mixing was continued until all solvent got evaporated. To the residue, distilled water was added and thoroughly mixed to achieve an emulsion. The emulsion was sprayed using spray dryer and powder was obtained.

### EXAMPLE 4

| Sr. No | Name of Ingredient | Qty for 100 g |
|---|---|---|
| 1 | Curcumin Extract | 11.52 |
| 2 | Green Tea Extract | 22.50 |
| 3 | Green Coffee Bean Extract | 22.50 |
| 4 | Ethanol | 129.0 |
| 5 | Methylene Dichloride | 300.0 |
| 6 | Modified Starch | 39.48 |
| 7 | Tween 80 | 4.00 |
| 8 | Purified Water | 100.0 |

11.52 g of curcumin extract was dissolved in mixture of 129.0 g ethanol and 300.0 g of methylene dichloride. 39.48 g modified starch and 4.00 g of Tween 80 were dissolved in 100 g of hot purified water at 50° C. The solvent phase was added slowly to the aqueous phase using both a high rate of mixing and a high shear force mixer. After the addition was completed, the emulsion temperature was maintained at 50° C. and 22.50 g of green coffee bean extract and 22.50 g green tea extract were added. High speed shear mixing was continued for 15 min. The temperature was gradually raised and mixing was continued until all solvent had evaporated. During the evaporation procedure, distilled water was added to the mixture to achieve an emulsion. This resulting emulsion was spray dried to get powder.

### EXAMPLE 5

| Sr. No | Name of Ingredient | Qty for 100 g |
|---|---|---|
| 1 | Curcumin Dispersible Powder 20% | 50.00 |
| 2 | Green Coffee Bean Extract | 50.00 |
| 3 | Purified Water | 400.00 |

50.0 g of curcumin dispersible powder 20% and 50.0 g of Green Coffee Bean Extract were dissolved in 400 ml of purified water under constant stirring for 30 min. The dispersion obtained was dried on a spray dryer to get granules.

### EXAMPLE 6

| Sr. No | Name of Ingredient | Qty for 100 g |
|---|---|---|
| 1 | Curcumin Dispersible Powder 40% | 25.00 |
| 2 | Green Tea Extract | 25.00 |
| 3 | Green Coffee Bean Extract | 25.00 |
| 4 | Modified starch | 25.00 |
| 5 | Purified Water | 400.00 |

25.0 g of curcumin dispersible powder 40%, 25.0 g of Green Coffee Bean Extract, 25.0 g of Green Tea Extract and 25.0 g of modified starch were dissolved in 400 ml of purified water under constant stirring for 30 min to obtain a dispersion. This dispersion was then sprayed using spray dryer to get final powder.

It will be readily apparent to one skilled in the art that varying substitutions and modifications may be made to the invention disclosed herein without departing from the spirit of the invention. Thus, it should be understood that although the present invention has been specifically disclosed by the preferred embodiments and optional features, modification and variation of the concepts herein disclosed may be resorted to by those skilled in the art, and such modifications and variations are considered to be falling within the scope of the invention.

It is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It must be noted that, as used in this specification and the appended claims, the singular forms “a,” “an” and “the” include plural references unless the context clearly dictates otherwise.

1. A phytoactive combination comprising:
plant extracts that deliver curcumin, chlorogenic acid, and/or catechin.

2. The phytoactive combination of claim 1, wherein the phytoactive combination increases bioavailability of the curcumin.

3. The phytoactive combination of claim 1, wherein the chlorogenic acid and/or the catechin combines with the curcumin synergistically, thereby improving therapeutic and pharmacological effects of the phytoactive combination.

4. The phytoactive combination of claim 1, wherein the phytoactive combination is delivered to a subject such that the subject receives a range of 10 milligrams to 500 milligrams per day of the curcumin, a range of 10 milligrams to 800 milligrams per day of the chlorogenic acid, and a range of 5 milligrams to 1,000 milligrams per day of the catechin.

5. The phytoactive combination of claim 1, wherein the phytoactive combination is dispensed in a dosage form suitable for oral delivery.

6. The phytoactive combination of claim 5, wherein the dosage form is selected from the group consisting of: powder, pellets, beadlets, granules, tablet, capsules, buccal films, and combinations thereof.

7. The phytoactive combination of claim 1, wherein the phytoactive combination is prepared by encapsulating the curcumin, the chlorogenic acid, and/or the catechin.

8. The phytoactive combination of claim 1, wherein the phytoactive combination is prepared by a process comprising combining the plant extracts with one or more encapsulating agents and one or more solvents.

9. The phytoactive combination of claim 8, wherein the phytoactive combination comprises active ingredients having a range of between 1% to 95% w/w and wherein the encapsulating agent has a range from between 5% and 90%.

10. The phytoactive combination of claim 1, wherein the phytoactive combination is used for a purpose selected from the group consisting of: enhancing endurance and stamina, significantly reducing cortisol levels, thereby reducing stress, elevating hydrolysis of adenosine triphosphate (ATP), improving blood flow, quick recovery from sports injury, better weight and fat loss compared to administration of individual ingredients of the phytoactive combination alone, increasing and strengthening muscles, increasing maximum oxygen uptake during incremental exercise, and combinations thereof.

11. The phytoactive combination of claim 9, wherein the active ingredients have a range of between 20% and 50% w/w and wherein the encapsulating agent has a range of between 30% and 70%.
